(19) United States
(12) Patent Application Publication
(10) Pub. No.: US 2025/0185908 A1
(43) Pub. Date: Jun. 12, 2025

(54) OCT SPECKLE VELOCIMETRY

(71) Applicant: TOPCON CORPORATION, Tokyo (JP)

(72) Inventors: Tony KO, Cupertino, CA (US); Xiang WEI, Cupertino, CA (US); Masahiro AKIBA, Saitama (JP); Jen-Wei KUO, Livingston, NJ (US); Reza JAFARI, Kinnelon, NJ (US); Richard SPAIDE, New York, NY (US)

(21) Appl. No.: 18/846,538

(22) PCT Filed: Mar. 14, 2023
(86) PCT No.: PCT/US2023/064299

§ 371 Date: Sep. 12, 2024

Related U.S. Application Data
(60) Provisional application No. 63/269,298, filed on Mar. 14, 2022.

(51) Int. Cl. A61B 3/10; A61B 3/12; G01P 5/26; G06T 7/00

(52) U.S. Cl. CPC A61B 3/102; A61B 3/1233; G01P 5/26; G06T 7/0016; G06T 2207/10101; G06T 2207/20024; G06T 2207/20056; G06T 2207/20081; G06T 2207/30041; G06T 2207/30104

(57) ABSTRACT

Blood flow information is extracted from speckle information of a time series of structural optical coherence tomography (OCT) images. Flow information can be determined from high-frequency information of the OCT images, a speckle density of the OCT images, from a co-occurrence matrix applied to the OCT image, from a machine learning analysis of an input OCT image, or the like. A flow profile analogous to a pulse waveform is then generated as a time series of the extracted flow information.

### CROSS-REFERENCE TO RELATED APPLICATIONS

This application claims priority to U.S. Provisional Patent Application 63/269,298 filed on Mar. 14, 2022, the entirety of which is incorporated herein by reference.

### BACKGROUND

Optical coherence tomography (OCT) is a non-invasive imaging technique, often used in ophthalmology. OCT relies on principles of interferometry to image and collect information about an object (such as the eye of a subject). Particularly, light from a source is split into a sample arm where it is reflected by the object being imaged, and reference arm where it is reflected by a reference object such as a mirror. The reflected lights are then combined in a detection arm in a manner that produces an interference pattern that is detected by spectrometer, photodiode(s) or the like. The detected interference signal is processed to reconstruct the object and generate structural OCT images.

More particularly, structural OCT images and volumes are generated by combining numerous depth profiles (A-lines, e.g. along a Z-depth direction at an X-Y location) into a single cross-sectional image (B-scan, e.g., as an X-Z or Y-Z plane), and combining numerous B-scans into a volume. These depth profiles are generated by scanning along the X and Y directions. En-face images in the X-Y plane may be generated by flattening a volume in all or a portion of the Z-depth direction, and C-scan images may be generated by extracting slices of a volume at a given depth. In other methods, OCT images are acquired in an en-face manner in the X-Y plane with the Z-depth acquired sequentially. Cross-sectional images in the X-Z or Y-Z planes may be generated from the acquired volume.

In some applications, OCT imaging may be used to determine blood flow properties, such as velocity. One technique for doing so is Doppler OCT, which measures the Doppler shifts caused when blood cells scatter the OCT light beam. However, Doppler OCT requires extracting phase information from raw spectral data and determining Doppler phase shifts. These processes can be complicated and difficult to efficiently perform.

### BRIEF SUMMARY

According to one example of the present disclosure, a method comprises: capturing optical coherence tomography (OCT) data from an object; generating a first plurality of structural OCT images from a first location of the object based on the captured OCT data; extracting flow information from individual ones of the first plurality of structural OCT images; and generating a first time-series flow profile of the first location of the object, wherein the first flow profile is a relationship between the extracted flow information and a timing of the captured OCT data from which the corresponding individual one of the first plurality of structural OCT images was generated.

In various embodiments of the above example, extracting flow information comprises applying a high frequency filter to frequency information of the individual one of the first plurality of structural OCT images, thereby producing high frequency information, and the flow information corresponds to the high frequency information; extracting flow information further comprises: applying a low frequency filter to the frequency information of the individual one of the first plurality of structural OCT images, thereby producing low frequency information, wherein the flow information is a ratio of the high frequency information to the low frequency information; extracting flow information comprises: applying two-dimensional Fourier transform to the individual one of the first plurality of structural OCT images, thereby producing the frequency information; the extracted flow information is a speckle density of the individual one of the first plurality of structural OCT images; extracting flow information comprises: applying a co-occurrence matrix to the first plurality of structural OCT images, and determining a correlation among the first plurality of structural OCT images based on the co-occurrence matrix; extracting flow information comprises: inputting the individual one of the first plurality of structural OCT images to a machine learning system trained to output flow information based on an input structural OCT image; extracting flow information and generating the time-series flow profile comprises: inputting the first plurality of structural optical coherence tomography (OCT) images as a time series to a machine learning system trained to output the flow profile based on an input time series of structural OCT images; the OCT data is captured for a time period comprising a plurality of cardiac cycles; the method further comprises: displaying the first flow profile as a time-series graph; the method further comprises: extracting the flow information from a plurality of regions of the individual one of the first plurality of structural OCT images, generating a flow map of the extracted flow information over the plurality of regions, and displaying the flow map; the method further comprises: generating the flow map for at least two of the first plurality of structural OCT images, generating a flow video from the generated flow maps, and displaying the flow video; the first location is a cross-sectional location and the OCT data is captured from the first cross-sectional location and from a second cross-sectional location a known distance from the first cross-sectional location, wherein the method further comprises: generating a second plurality of structural OCT images from the second cross-sectional location of the object, generating a second time-series flow profile of the second cross-sectional location of the object, determining a time difference between the first flow profile and the second flow profile, and determining a flow velocity of the object based on the known distance and the determined time difference; the OCT data is alternately captured between the first cross-sectional location and the second cross-sectional location; the time difference is between local maxima or local minima of the first and second flow profiles; the method further comprises: applying a stimulus to the object, and determining a change to the first flow profile in response to application of the stimulus; the applied stimulus is pressure; the flow information is extracted from a region of interest identified in one of the first plurality of structural OCT images, and which is registered to the other first plurality of structural OCT images; the region of interest corresponds to an area of blood flow, and is automatically identified; and/or the object is an eye.

### BRIEF DESCRIPTION OF THE SEVERAL VIEWS OF THE DRAWING

FIG. 1 illustrates an example optical coherence tomography (OCT) system.

FIG. 2 illustrates an example method according to the present disclosure.

FIG. 3 illustrates an example method for extracting blood flow information based on frequency.

FIG. 4A illustrates a first example method for extracting blood flow information based on speckle density.

FIG. 4B illustrates a second example method for extracting blood flow information based on speckle density.

FIG. 5 illustrates an example method for extracting blood flow information based on a co-occurrence matrix.

FIG. 6A comparatively illustrates a flow profile determined according to the present disclosure with a corresponding structural OCT signal from which the flow profile was determined.

FIG. 6B comparatively illustrates a blood flow map and structural OCT image for the frames identified in FIG. 6A.

FIG. 7 illustrates an example method for determining flow velocity.

FIG. 8A illustrates an en-face image identifying scan locations for determining a flow velocity.

FIG. 8B illustrates flow profiles of the locations indicated in FIG. 8A, and the determination of flow velocity therefrom.

FIG. 9 illustrates an example method of glaucoma testing.

### DETAILED DESCRIPTION OF THE DRAWING

Based on the foregoing deficiencies, the present disclosure relates to determining blood flow information from optical coherence tomography (OCT) images without the need for complex processing. More particularly, the present disclosure relates to systems and methods for determining blood flow information from structural OCT images without a Doppler or like phase-related analysis. Still more particularly, the present disclosure determines blood flow information based on speckle information in the structural OCT images.

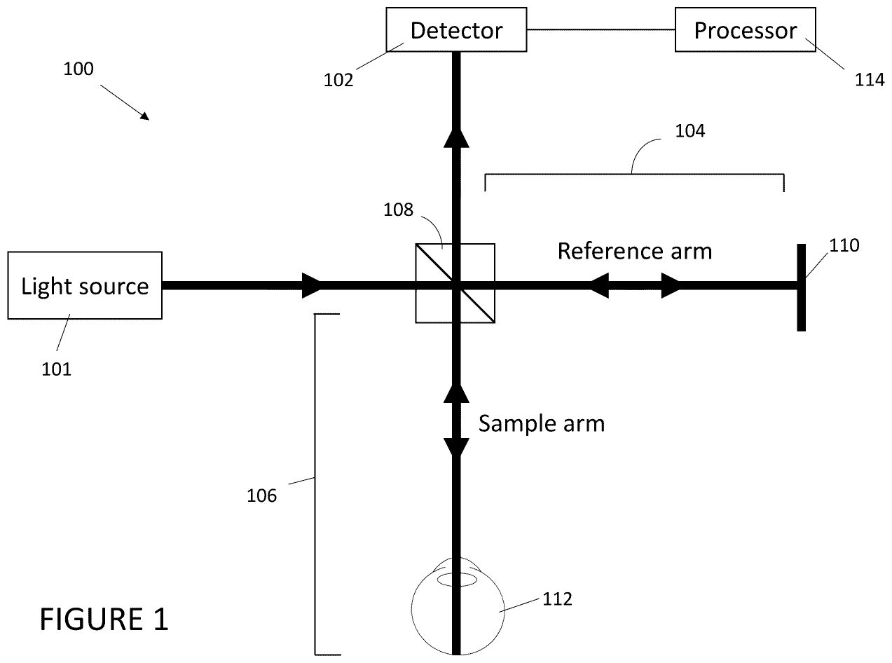

An example OCT system 100 such as that of the present disclosure is illustrated in FIG. 1. As discussed above the system 100 includes a light source 101. The light generated by the light source 101 is split by, for example, a beam splitter (as part of interferometer optics 108), and sent to a reference arm 104 and a sample arm 106. The light in the sample arm 106 is backscattered or otherwise reflected off an object, such as the retina of an eye 112. The light in the reference arm 104 is backscattered or otherwise reflected, by a mirror 110 or like object. Light from the sample arm 106 and the reference arm 104 is recombined at the optics 108 and a corresponding interference signal is detected by a detector 102. Herein, the “speckle” of an OCT image is understood to mean the characteristic interference pattern produced by the backscattered OCT light from a turbid media such as tissue microstructure. The detector 102 can be a spectrometer, photo detector, or any other light detecting device. The detector 102 outputs an electrical signal corresponding to the interference signal to one or more processors 114. The processor(s) 114 may process the electrical signal into OCT signal data, generate corresponding structural images, and/or further analyze the data and images. Particularly, the processor(s) 114 may implement any or all aspects of the present disclosure.

The processor(s) 114 may also be associated with an input/output interface (not shown) including a display for outputting processed images, or information related to the analysis of those images. The input/output interface may also include hardware such as buttons, keys, or other controls for receiving user inputs to the system. In some embodiments, the processor 114 may also be used to control the light source and imaging process.

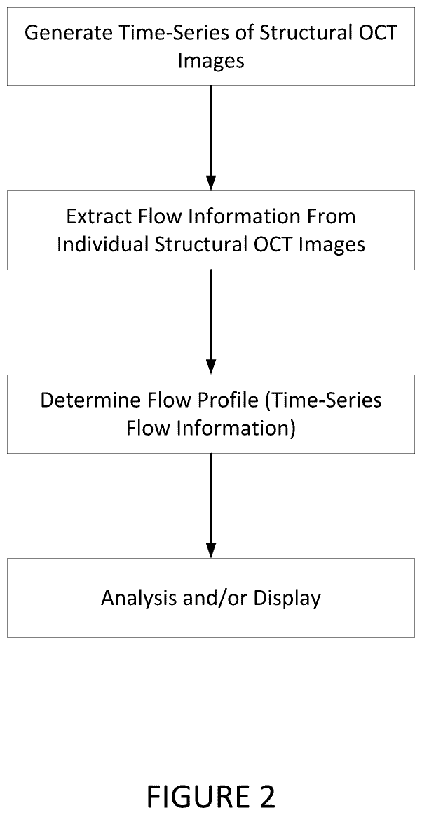

An example method of the present disclosure is illustrated generally in FIG. 2. Therein, the method begins by generating a time-series of OCT structural images at a location of the object being imaged. In other words, the OCT system 100 of FIG. 1 images the object according to a scan pattern that produces OCT data from the same location at a plurality of times. For example, the OCT system 100 may repeatedly scan a single cross-sectional plane location of the object so as to generate a plurality of B-scans of the cross-sectional plane location at different times. In other embodiments, the OCT system 100 may repeatedly scan an area of the object so as to generate a plurality of volumes of the object, from which B-scans at different times can be derived.

For analyzing blood flow, the scanning protocol of the OCT system 100 may collect data for a long enough period of time to capture one or more cardiac pulses. In other words, the OCT data may be collected for a time on the order of a few seconds. According to one example, OCT data is collected for between two and three seconds. Further, the rate and density of the OCT data capture may be oversampled to improve the amount of speckle information in each resulting structural OCT image. For example, the OCT system 100 may operate at a frequency great enough to obtain at least 50 repeated structural OCT B-scans per second at a common cross-sectional plane location. According to one example, each structural OCT B-scan is about 1 mm wide with between 500 to 1024 A-lines of information.

During OCT data capture, the object may be tracked to ensure movement is limited during the capture period. If movement is too great, thereby causing too much noise or an inability to register the resulting OCT B-scans or volumes, the scanning protocol may be reset.

Following OCT data capture and structural image generation, blood flow information is extracted from the individually generated structural OCT images. Depending on the embodiment, blood flow information may be extracted from an entire OCT B-scan, or only from a region of interest (ROI). If extracted from an ROI, the ROI may be determined manually or automatically. The ROI may be manually determined by a clinician selecting a region of an OCT B-scan corresponding to an area of blood flow or vasculature. Measurements of the vasculature, such as its size, may also be measured manually by a clinician from the structural OCT images. Such a region and measurements may instead be automatically determined by a variety of methods. For example, the region of vasculature (and thus blood flow) may be identified by segmentation techniques, machine learning techniques, thresholding techniques (blood flow and vasculature having a greater intensity and variation), and the like. Further, additional ROIs may be identified for other vessels.

As noted above, each of the repeated B-scans at the common cross-sectional plane location may be registered to each other. Therefore, the ROI may only be identified in a B-scan image and then extrapolated to the other repeated B-scan images at that location. In the event that images are acquired in an en-face manner in the X-Y plane, those X-Y plane images may also be registered to each other. Thus, the ROI would only need to be identified in a single X-Y plane image.

Blood flow information may be extracted from individual OCT B-scans in a variety of ways. Generally, the techniques for extracting blood flow information from structural OCT B-scans are based on the recognition that blood flow produces greater speckle variation than static structural tissue.

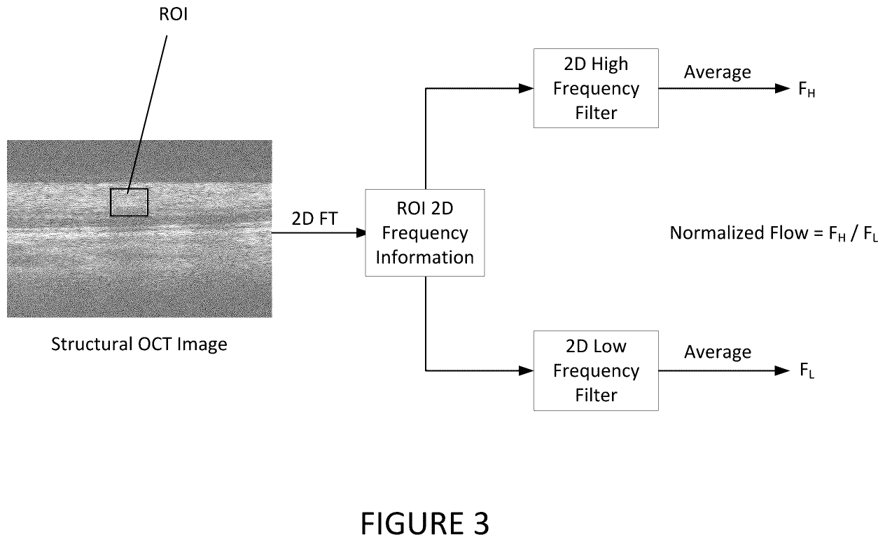

A first example method for extracting blood flow information based on frequency analysis is illustrated in FIG. 3. As seen therein, an ROI is identified within an area of an OCT structural image corresponding to blood flow and vasculature (as indicated by the higher intensity signal and higher density speckle pattern). The ROI is then transformed (e.g., according to a 2D Fourier transform) to produce 2D frequency information of the ROI, which may then be frequency filtered. In other words, high and low frequency filters are applied to frequency information of the transformed ROI. The cutoff frequency of the filters may be determined based on a size of the ROI (or 2D Fourier transform region) in relation to the size of blood vessel speckles under analysis. The resulting signal is then statistically combined into a single value to produce a high frequency signal F_{H}, and a low frequency signal F_{L}. According to the example of FIG. 3, the statistical combination is an average.

Generally, the high frequency signal F_{H} can be understood to represent blood flow, while the low frequency signal F_{L} represents static tissue. Therefore, in some embodiments, the high frequency signal F_{H} alone may be determined and further processed, and correspond to the flow information. However, in some embodiments, normalizing the high frequency signal F_{H} (for example, to the low frequency signal F_{L}) may help account for natural variations in the imaged object and the OCT system 100, thereby improving the quality of the extracted flow information. In other words, the flow information may be determined as the ratio between the high and low frequency signals F_{H}, F_{L}.

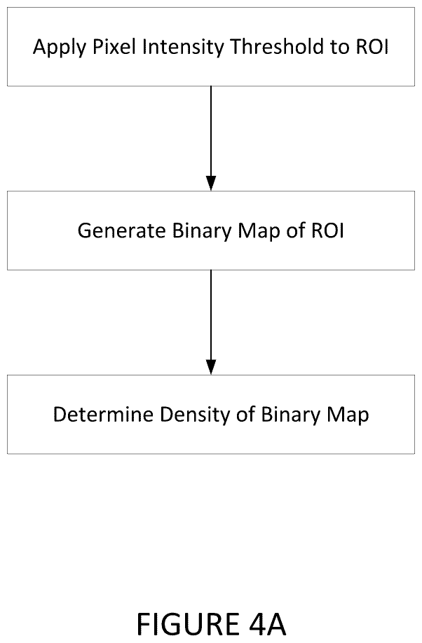

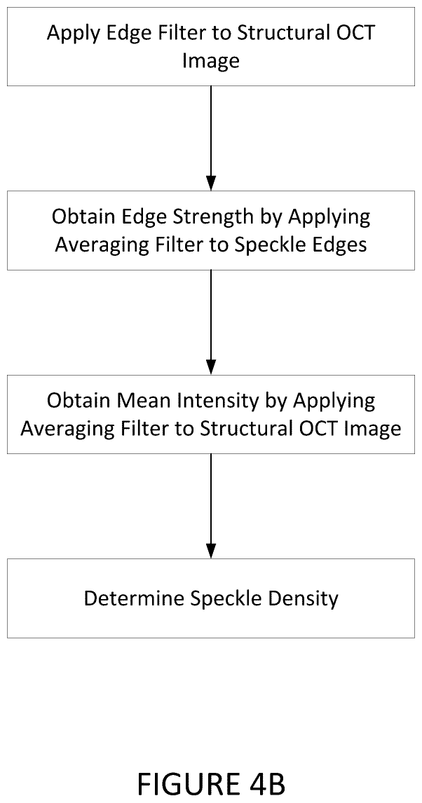

According to another technique, flow information may be extracted based on a speckle density. A first example of such a technique is illustrated in FIG. 4A. According to the example of FIG. 4, a threshold may be applied to the pixels within the ROI. A binary map of the ROI may then be generated based on an applied threshold. For example, all pixels greater than the threshold may be set to a value of 1, while all pixels less than threshold may be set to a value of 0. A speckle density may then be determined as a ratio of the number of pixels having a value of 1 to the total number of pixels of the ROI, or to the number of pixels having a value of 0. The flow information may thus be considered equal to or a function of speckle density.

A second example technique for determining speckle density is illustrated in FIG. 4B. According to the method therein, an edge filter is first applied to a structural OCT B-scan, which identifies edges of the speckles. An averaging filter is then applied to the speckle edges (the output of the edge filter) and to the structural OCT B-scan. Applying the averaging filter produces an edge strength and a mean intensity of the structural OCT B-scan, respectively. The averaging filter preferably has size that is close to the size of the blood vessel in the structural OCT B-scan from which the blood flow information is being determined. Finally, because speckle density is proportional to the density of visible edges, the speckle density can be determined as a ratio between the edge strength and the mean intensity (e.g., edge strength divided by mean intensity). As above, the flow information may be considered the speckle density (e.g., the ratio) or a function of the speckle density.

In still other embodiments, speckle density could be determined by applying a threshold intensity to the speckle edges. In other words, the speckle density may be considered the number of speckle edges exceeding a threshold intensity. However, normalizing (e.g., to the mean intensity as discussed above) helps retain edge strength information at different locations.

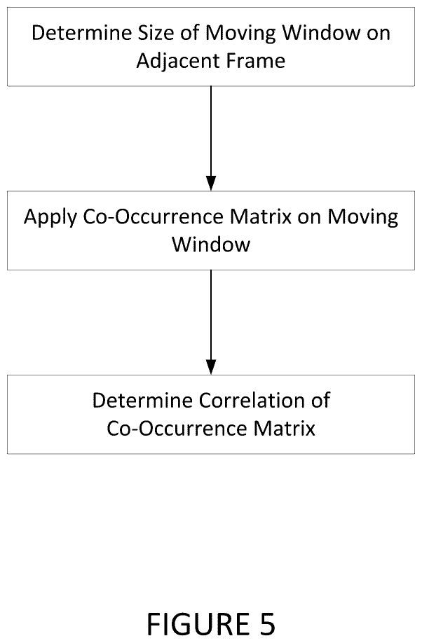

In still other techniques, flow information may be determined by applying a co-occurrence matrix to the ROI. The co-occurrence matrix creates a dependence matrix by determining how often a pixel with pixel intensity value i occurs in the adjacent frame to a pixel with the value j. Each element (i, j) in the dependence matrix specifies the number of times that the pixel with a value i occurred in the adjacent frame to a pixel with value j. The flow information is then determined by applying the correlation of a pixel to its neighbor frames on the co-occurrence matrix. An example application of such a co-occurrence matrix application is illustrated in FIG. 5. The size of a moving window applied on temporally adjacent structural OCT B-scan images is first determined. According to one example, the moving window size is four. A co-occurrence matrix is then applied to the structural OCT B-scan image data inside this analysis window. The flow information is then determined by applying a correlation property on the co-occurrence matrix.

In still other techniques, flow may be determined by a machine learning system. Such a machine learning system may be trained to output one or more values representing flow information based on an input structural ROI, OCT B-scan, or OCT volume. For example, the machine learning system may be trained in a supervised manner based on input structural OCT image information and a corresponding ground truth flow information. The ground truth flow information may be determined, for example, according to one of the above-described techniques. The ground truth flow information may additionally or alternatively be determined by other techniques, such as Doppler OCT or even non-OCT analysis techniques. Accordingly, the machine learning system is trained to recognize a relationship between a structural speckle signal and corresponding flow information.

In still other techniques, flow may be determined by applying known ultrasound and laser speckle techniques, such as speckle decorrelation, speckle contrast, and speckle auto-correlation. Each of these techniques could be applied on individual structural OCT B-scans and/or on temporally adjacent structural OCT B-scan images. In still other techniques, flow may be determined by applying known amplitude-based OCT angiography techniques such as speckle variance, amplitude-decorrelation, or split-spectrum amplitude-decorrelation. As above, each of these techniques could be applied on individual structural OCT B-scans and/or on temporally adjacent structural OCT B-scan images.

Returning to FIG. 2, once flow information is identified for a plurality of repeated OCT B-scan images (or other images at a common location), a flow profile may be determined as a time-series plot of the flow information. In other words, the ratio between the high and low frequency signals F_{H}, F_{L}, the speckle density, or like flow-related information values are plotted according to a time series in which the relative time for each flow-related information value corresponds to a time at which the corresponding repeated OCT image was captured.

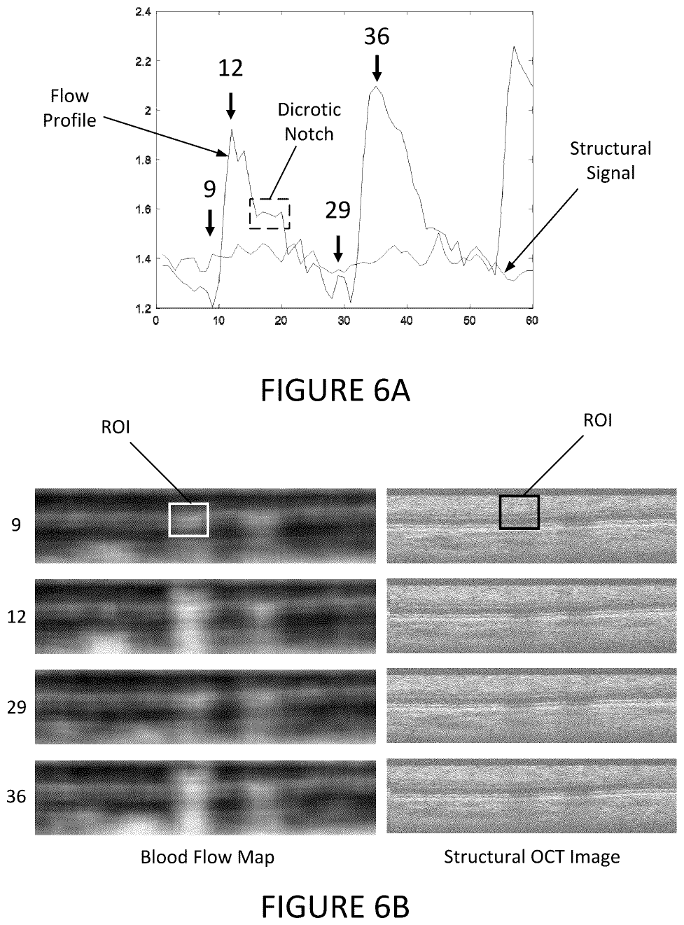

An example flow profile is illustrated in FIG. 6A along with a corresponding structural OCT signal. The structural OCT signal represents an average pixel intensity within the ROI form which the flow information of the flow profile was extracted. As can be seen, the flow profile is similar to a traditional pulse waveform. Indeed, the dicrotic notch is even visible roughly between image frames 15 and 20. In contrast, the structural OCT signal is relatively flat and bears no relationship to a pulse wave.

FIG. 6A further identifies four image frames (9, 12, 29, and 36) which are illustrated in FIG. 6B. More particularly, FIG. 6B illustrates the original structural OCT image for each frame along with a blood flow map for each frame. The blood flow map is a map of extracted flow information for the entire area of the structural OCT image, while the time-series signals shown in FIG. 6A only represent the ROI noted in the image frames in FIG. 6B.

Comparing the flow profile in FIG. 6A with the blood flow maps of FIG. 6B, the pixel intensity (and thus the extracted blood flow information) of the area corresponding to the ROI is relatively low (dark) in frames 9 and 29, but relatively high (bright) in frames 12 and 36. In contrast, there is little-to-no discernible difference in the ROI in the structural OCT images at any frame.

In some embodiments, a flow profile may be generated by a machine learning system. For example, a machine learning system may be trained to output a flow profile based on an input time-series of structural OCT images.

While the above description relates to generating flow profiles at a common cross-sectional plane location, it is possible to determine a flow velocity based on flow profiles from two different cross-sectional plane locations.

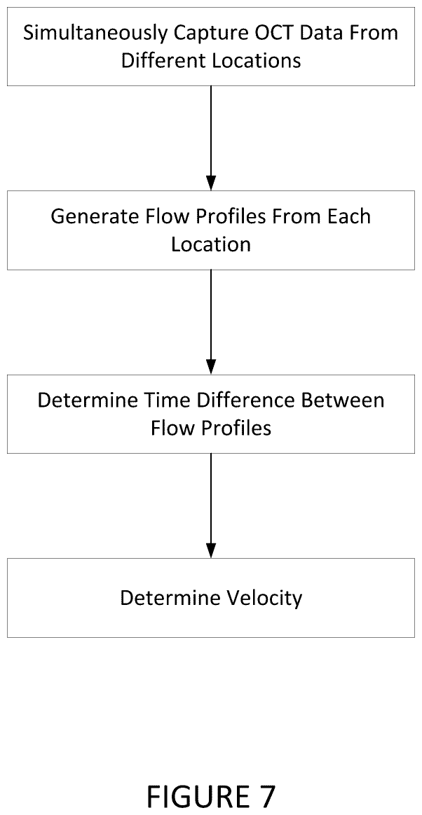

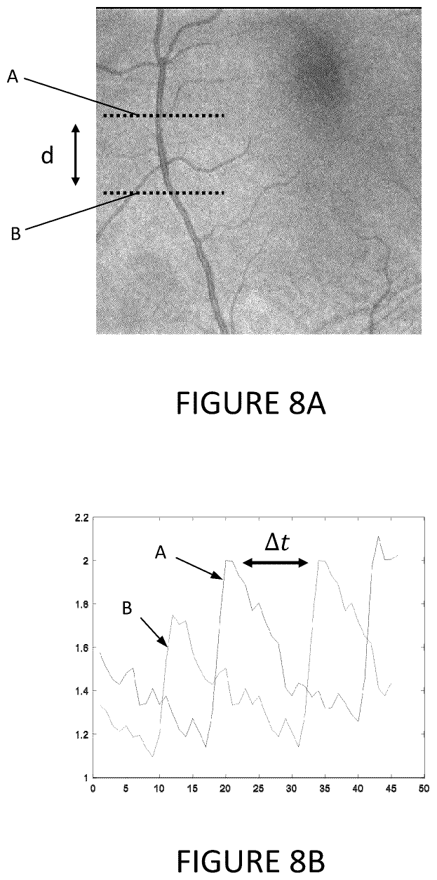

With reference to FIG. 7, the first step of an example method for determining flow velocity involves simultaneously capturing OCT data from at least two different locations having a known distance therebetween. According to one example, the OCT data capture occurs alternately between the different locations such that the resulting B-scan images from each location are temporally interleaved. FIG. 8A illustrates an en-face image in which two locations A, B are identified along a blood vessel therein. The locations A, B are separated by a distance d. In some embodiments, OCT data may be captured according to circular scans having different radii. The difference in radius between each circular scan thus represents a known distance. These circular scans could be temporally interleaved in the same manner discussed above.

Following the capture of OCT data, flow profiles for the data from each location are generated. These flow profiles may be generated from any of the above-discussed methods. FIG. 8B illustrates flow profiles corresponding to the locations A, B of FIG. 8A. A time At between these two flow profiles can then be determined. For example, as illustrated in FIG. 8B, the time At is determined between local maxima of the flow profiles; however, the time may instead be determined between other common locations of the flow profiles (e.g., local minima). The local maxima may be determined, for example, by identifying the frame at which a maximum value in each cardiac cycle occurs. According to other examples, a derivative of the flow profile may be determined and then the frames at which the derivative equals (or crosses) zero correspond to local maxima and minima. The time At may then be determined based on the sampling rate at which OCT data was captured. In other words, if OCT B-scans are captured at 50 frames per second, a difference of 50 frames between local maxima of the different flow profiles represents a 1 second difference.

Once the time Δt is determined, a velocity may be determined according to the relationship velocity=d/Δt. A direction of the flow may be further determined according to whether a phase shift of the OCT signal between the two locations is positive for negative. This velocity may be determined multiple times between the same two locations and/or between multiple locations. The plurality of determined velocities may then be averaged (or combined according to another statistical determination) in order to identify a representative velocity. Similarly, determined velocities can be compared between a plurality of patients to identify abnormalities, or between the same patient at multiple capture times to determine a change in the patient's condition.

Still further, any part of the above description may be incorporated with other analysis techniques and/or processing. For example, the flow profiles may be averaged over several cardiac cycles or compared over a period of time (e.g., weeks, months, years) to monitor disease progression. In other examples, because blood flow in veins and arteries may be considered opposite, the determined distance of blood flow may be used to distinguish arteries from veins in the structural images. Similarly, because blood flow is slower in veins than arteries, veins and arteries could further be distinguished by comparison of determined velocity. In still other examples, the above OCT data acquisition could be performed concurrently with other physiological measurements, such as pulse oximetry, electrocardiogram and the like. Further analysis of the flow profiles herein may be based on additional physiological information captured by the concurrent measurements.

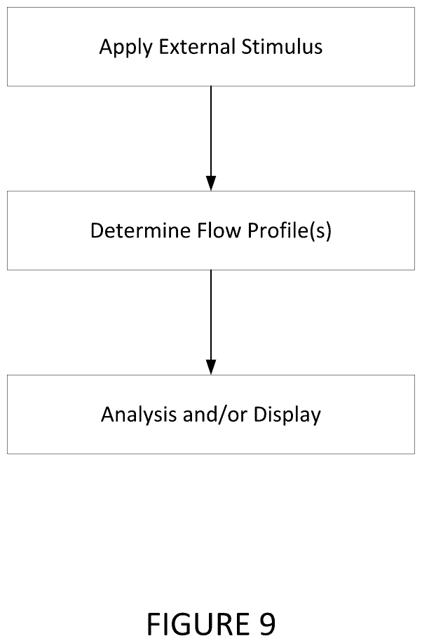

Additionally, any vascular biomarkers of glaucomatous damage such as impaired vascular flow and autoregulation may be used to provide diagnostic information. Accordingly, it may be possible to detect glaucoma, or quantify its severity, by measuring changes in blood flow in response to an external stimulus. An example of such a method is illustrated in FIG. 9.

As seen in the example method of FIG. 9, an external stimulus is applied. In the example of glaucoma testing, such a stimulus may be an applied pressure (e.g., an air puff) or other stimulus that produces a change in intraocular pressure. A flow profile may then be determined for the patient according to any of the above techniques. The resulting flow profile may be compared to known healthy patients or a previously determined baseline flow profile for that patient. Changes in the flow profile relative to the baseline may be indicative of a healthy eye, as flow in the eye (and thus the vessels of the eye) are affected by the applied pressure. In contrast, no (or relatively minimal) change in flow profile relative to the baseline may be indicative of glaucoma since the blood flow and vessel are not responsive to the applied pressure. The above analysis may also be applied vice versa depending on the applied external stimulus.

While the above example relates to an applied pressure for testing glaucoma, it should be understood that any external stimulus and testing could be utilized. In other words, a flow profile may be determined according to the present disclosure before, during, or after the application of any external stimulus. Response of the flow profile to the stimulus may then be analyzed for diagnostic or like purposes.

Returning again to FIG. 2, with the flow profile known, the extracted flow information may be further analyzed and/or displayed. Because the above-discussed flow profile corresponds to a pulse waveform, such an analysis can include any known techniques for analyzing pulse waveforms. Such techniques may include those for determining heart rate, rise time, flow skew, blood pressure, cardiac function, vessel stiffness, and the like.

Regarding displays, display of the extracted flow information may include the flow profile itself, the blood flow maps, and/or the structural OCT images. The extracted flow information may be mapped to pixel intensity and/or color in the blood flow maps. In some embodiments, the blood flow maps may be shown as videos rather than static images.

While various features are presented above, it should be understood that the features may be used singly or in any combination thereof. Further, it should be understood that variations and modifications may occur to those skilled in the art to which the claimed examples pertain.

What is claimed is:

1. A method comprising:
capturing optical coherence tomography (OCT) data from an object;
generating a first plurality of structural OCT images from a first location of the object based on the captured OCT data;
extracting flow information from individual ones of the first plurality of structural OCT images; and
generating a first time-series flow profile of the first location of the object,
wherein the first flow profile is a relationship between the extracted flow information and a timing of the captured OCT data from which the corresponding individual one of the first plurality of structural OCT images was generated.

2. The method of claim 1,
wherein extracting flow information comprises applying a high frequency filter to frequency information of the individual one of the first plurality of structural OCT images, thereby producing high frequency information, and
wherein the flow information corresponds to the high frequency information.

3. The method of claim 2, wherein extracting flow information further comprises:
applying a low frequency filter to the frequency information of the individual one of the first plurality of structural OCT images, thereby producing low frequency information,
wherein the flow information is a ratio of the high frequency information to the low frequency information.

4. The method of claim 2, wherein extracting flow information comprises:
applying two-dimensional Fourier transform to the individual one of the first plurality of structural OCT images, thereby producing the frequency information.

5. The method of claim 1, wherein the extracted flow information is a speckle density of the individual one of the first plurality of structural OCT images.

6. The method of claim 1, wherein extracting flow information comprises:
applying a co-occurrence matrix to the first plurality of structural OCT images; and
determining a correlation among the first plurality of structural OCT images based on the co-occurrence matrix.

7. The method of claim 1, wherein extracting flow information comprises:
inputting the individual one of the first plurality of structural OCT images to a machine learning system trained to output flow information based on an input structural OCT image.

8. The method of claim 1, wherein extracting flow information and generating the time-series flow profile comprises:
inputting the first plurality of structural optical coherence tomography (OCT) images as a time series to a machine learning system trained to output the flow profile based on an input time series of structural OCT images.

9. The method of claim 1, wherein the OCT data is captured for a time period comprising a plurality of cardiac cycles.

10. The method of claim 1, further comprising:
displaying the first flow profile as a time-series graph.

11. The method of claim 1, further comprising:
extracting the flow information from a plurality of regions of the individual one of the first plurality of structural OCT images;
generating a flow map of the extracted flow information over the plurality of regions; and
displaying the flow map.

12. The method of claim 11, further comprising:
generating the flow map for at least two of the first plurality of structural OCT images;
generating a flow video from the generated flow maps; and
displaying the flow video.

13. The method of claim 1, wherein the first location is a cross-sectional location and wherein the OCT data is captured from the first cross-sectional location and from a second cross-sectional location a known distance from the first cross-sectional location, the method further comprising:
generating a second plurality of structural OCT images from the second cross-sectional location of the object;
generating a second time-series flow profile of the second cross-sectional location of the object;
determining a time difference between the first flow profile and the second flow profile; and
determining a flow velocity of the object based on the known distance and the determined time difference.

14. The method of claim 13, wherein the OCT data is alternately captured between the first cross-sectional location and the second cross-sectional location.

15. The method of claim 13, wherein the time difference is between local maxima or local minima of the first and second flow profiles.

16. The method of claim 1, further comprising:
applying a stimulus to the object; and
determining a change to the first flow profile in response to application of the stimulus.

17. The method of claim 16, wherein the applied stimulus is pressure.

18. The method of claim 1, wherein the flow information is extracted from a region of interest identified in one of the first plurality of structural OCT images, and which is registered to the other first plurality of structural OCT images.

19. The method of claim 18, wherein the region of interest corresponds to an area of blood flow, and is automatically identified.

20. The method of claim 1, wherein the object is an eye.
